(19) United States
(12) Patent Application Publication
(10) Pub. No.: US 2006/0045856 A1
(43) Pub. Date: Mar. 2, 2006

(54) Composition containing a dihydroxyacetone precursor

(76) Inventors: Teresa Mujica, Darmstadt (DE); Christophe Carola, Langen (DE); Sylvia Huber, Darmstadt (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/930,778

(22) Filed: Sep. 1, 2004

(51) Int. Cl. A61K 8/49

(52) U.S. Cl. 424/59

(57) ABSTRACT

The invention relates to a cosmetic or dermatological composition containing at least one precursor of a substance whose active form is sought for its cosmetic activity. One of the applications of the invention relates to compositions capable of rapidly imparting to the skin a color similar to that obtained on prolonged exposure to ultraviolet, solar or artificial radiation, while at the same time avoiding the drawbacks of such an exposure (erythema, burning, loss of elasticity, appearance of wrinkles, premature ageing of the skin, and the like).

The invention relates to a cosmetic or dermatological composition containing at least one precursor of a substance whose active form is sought for its cosmetic activity. One of the applications of the invention relates to compositions capable of rapidly imparting to the skin a color similar to that obtained on prolonged exposure to ultraviolet, solar or artificial radiation, while at the same time avoiding the drawbacks of such an exposure (erythema, burning, loss of elasticity, appearance of wrinkles, premature ageing of the skin, and the like).

For many years, the prior art has taught of the involvement of dihydroxyacetone (DHA hereinafter in the text) in the artificial coloring, of the skin (Bobin et al, J. Soc. Cosmet. Chem., 35 pages 265-272, 1984). DHA reacts with the amino acids naturally contained in the lipid film of the stratum corneum, and forms melanoids via a Maillard reaction (Maillard L. C., C. R. Acad. Sci. 154, 66-68, 1912).

Cosmetic compositions used for the purpose of artificially coloring the skin and containing DHA are widely described in the prior art.

In order to improve the effects of DHA, it is often combined with other substances for the purpose of increasing the speed of appearance of the color or the resistance of the latter over time. The combinations described in applications WO-A-9404130 or EP-A-547,864 may be mentioned, for example.

The use of DHA has a number of drawbacks which are quite incompatible with customer appeal. Indeed, the stability of DHA in formulation is entirely relative, causing degradation of the compound over time. It is observed in particular that compositions containing DHA sometimes acquire, before use, a color which users find quite unpleasant. In addition, a nauseating and unpleasant odor which consumers generally find undesirable may develop over time with these same compositions. The pH of the composition also decreases over time, which in the long run makes the composition incompatible with use in topical application.

Moreover, taking only the activity of DHA into consideration, it is known that its remanence on the skin is not perfect.

In order to solve these drawbacks, U.S. Pat. No. 5,693,670 proposes a novel cosmetic composition, one of the aims of which is to provide DHA at the time of application of the latter, in particular to the skin, without, however, the said composition containing DHA as such. The cosmetic or dermatological composition contains, in a cosmetically or dermatologically acceptable vehicle, at least one esterified dihydroxyacetone derivative corresponding to the general formula: R—O—CH₂—C(═O)—CH₂—O—R′ in which R and R′ represent a hydrogen atom or a saturated or unsaturated, linear, branched or cyclic, optionally hydroxylated acyl radical having from 2 to 25 carbon atoms, it being possible for R and R′ to be identical or different on condition that they are never simultaneously a hydrogen atom.

Despite these precursors of DHA have been synthetisized, there is still a demand for new self-tanning compounds with additional benefits in the cosmetic and dermatological industry.

The present invention relates to the use in the cosmetic and dermatology of compounds of formula I as precursors of dihydroxyacetone. In particular the application of these compounds as self-tanning actives in cosmetic and dermatological compositions. The present invention provides cosmetic and dermatological effective compositions, which comprise compounds of formula I 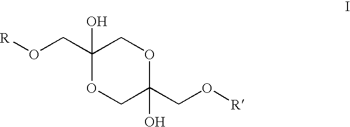
in which R and R′ represent a hydrogen atom or a saturated or unsaturated, linear, branched or cyclic acyl radical having from 2 to 25 carbon atoms, it is possible for R and R′ to be identical or different on condition that they are never simultaneously a hydrogen atom.

Compounds of formula I are dimeric molecules consisting of two monoester derivatives of dihydroxyacetone (DHA) covalent linked. The synthesis of compounds according to formula I is carried out using dihydroxyacetone and fatty acids or fatty acids chlorides. Standard procedures for the condensation of an alcohol with an acid or acid chloride to produce esters according to Can. J. of Chem, 1969, 1249—which is enclosed herein by reference—can be applied.

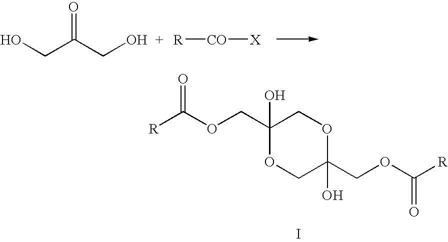

Under the reaction conditions, compound according to formula I can be directly prepared.

Prolonged heating of I in ethanolic solution yields the monomeric form, which slowly reverts to I by standing in the crystalline state at room temperature.

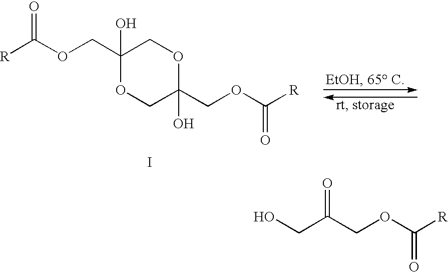

The advantages of using compounds according to formula I or compositions comprising these compounds are summarized below:
The skin penetration of compounds I have been enhanced due to the presence of fatty acid moieties.
Compounds I will be hydrolyzed to DHA by enzymes linked to the fat metabolism present in the skin.
Compounds I do not develop a tan as fast as DHA. The tanning process takes longer (˜3 days). The combination of DHA and I in formulations will successfully make the tan last longer.
Compounds I are solids and easy to formulate.

The invention as described above makes it possible to improve the remanence of the product applied as well as its stability in formulation. It nevertheless retains that one of the main advantages sought for such a cosmetic or dermatological composition, namely to use DHA for its properties, can only be achieved if the esterified derivative is converted into DHA at the moment of application. Such a hydrolysis, which at the moment of application releases DHA, is only possible in the presence of one (or more) compounds) capable of cleaving the ester bond(s) of the derivative. Such compounds are found on the skin. However, in order to improve the effectiveness of the composition according to the invention, it is in one embodiment of the invention desirable simultaneously to provide the esterified DHA derivative and the compound capable of cleaving an ester bond.

Therefore, preferred compositions comprise at least one compound capable of cleaving at least one ester bond.

This composition may advantageously be in a cosmetically or dermatologically acceptable form, for the purpose of its use in the fields in question.

According to a preferred embodiment of the invention, the acyl radical has from 3 to 18 carbon atoms.

The esterified derivative may advantageously be chosen from the group consisting of: 2-oxopropyl 1,3-didodecanoate, 2-oxopropyl 1,3-dihexadecanoate, 2-oxopropyl 3-hexadecanoate.

According to a preferred embodiment of the invention, the esterified derivative may be at a concentration ranging from 0.1% to 20% and preferably at a concentration ranging from 0.5% to 10%.

Here and in the remainder of the text, the percentages are given by weight relative to the total weight of the composition.

According to a particular embodiment of the latter, the compound capable of cleaving at least one ester bond may be any nucleophilic compound acceptable in cosmetics. Thus, alcohols, thiols, amines or anions may be mentioned.

Among the amines, a hydroxylated amine such as, for example, 3-amino-1,2-propanediol, 2-amino-2-methyl-1,3-propanediol, 2-amino-2-methylpropanol, 2-amino-2-hydroxymethyl-1,3-propanediol, glucamine or N-methylglucamine, or an amino acid such as, for example, lysine, arginine or histidine, will preferably be chosen.

Among the anions, a carboxylate anion such as, for example, fatty acid salts, amino acid salts or lipoamino acid salts will preferably be chosen.

Enzymes are the second large family of compounds which are capable of cleaving at least one ester bond and which may be used in the invention. Examples which may be mentioned are hydrolases, among which there will be mentioned, in a non-limiting manner, lipases, esterases or proteases. In the lipases, a pig pancreatic lipase such as that sold under the name Type II by the company Sigma or alternatively lipolase SP 644 sold by the company Novo-Nordisk will preferably be chosen.

According to the second object of the invention, and in another particular embodiment of the latter, the compound capable of cleaving at least one ester bond may be used at a concentration ranging from 0.1% to 30% and preferably ranging from 0.5% to 15%.

It is, however, preferable in the common use of such compositions to arrange matters such that hydrolysis of the esterified DHA derivative takes place only at the moment of application of the compositions. It is thus advantageous to provide packaging such that the esterified derivative and the compound capable of cleaving at least one ester bond are packaged so as not to be in contact with each other.

A further object of the invention thus relates to a composition in which the esterified derivative and the compound capable of cleaving at least one bond may be packaged so as not to be in contact with each other.

The two separate compartments joined together may, for example, constitute a single packaging in the form of a flexible tube, such that the esterified derivative and the compound capable of cleaving at least one ester bond are mixed together only when each of them is expelled from its own compartment. This expulsion may or may not be simultaneous. Mixing will take place during application.

Another form of packaging with two separate compartments joined together may be such that the esterified derivative or the compound capable of cleaving an ester bond is encapsulated in the form of microcapsules, spherules or any other form known to those skilled in the art, and is packaged in the presence of the other component of the invention in a different form, which is itself known to those skilled in the art.

In a cosmetic or dermatological form of this packaging, the non-encapsulated part of the invention may be a cream, a gel or any other form known to those skilled in the art.

There is nothing to prevent the two components from each being encapsulated. Here also, those skilled in the art know how to prepare such forms.

The release will take place during application, by crushing of the capsules under pressure exerted by the user, and the compositions thus released will be mixed together.

Regardless of the embodiment of the invention, in a cosmetic or dermatological application, it may also contain any other cosmetically or dermatologically acceptable constituent usually used in this type of composition, and in particular additives used to increase the effectiveness of the DHA originating from hydrolysis of the derivative.

The present invention further provides formulations comprising at least one self-tanning substance according to formula I, characterized in that the formulation comprises at least one fatty carrier and at least one hydrophilic solvent.

For the purposes of the present invention the formulations may contain additional self-tanning substances or self-tanning agents. Suitable additional self-tanning substances or self-tanning agents are all substances or mixtures of substances which are able to tan human skin without the effect of UV radiation. Advantageous self-tanning agents which may be used for the purposes of the present invention as additional self-tanning agents are the following substances:

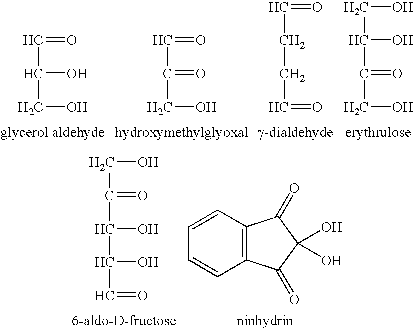

Mention is also made of 5-hydroxy-1,4-naphthoquinone (juglone), which is extracted from the shells of fresh walnuts 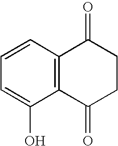
5-hydroxy-1,4-naphthoquinone (juglone)
and the 2-hydroxy-1,4-naphthoquinone (lawsone) which occurs in henna leaves. 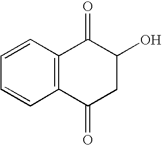
2-hydroxy-1,4-naphthoquinone (lawsone)

The most important active ingredient for self-tanning according to the present invention is 1,3-dihydroxyacetone (DHA), a trivalent sugar which occurs in the human body. 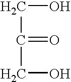
1,3-dihydroxyacetone (DHA)

The concentration of the at least one additional self-tanning substance preferred according to the invention is in the range from 0.01 to 15 percent by weight, preferably in the range from 0.05 to 5 percent by weight and particularly preferably at most 1% by weight.

The addition of hydrophilic solvents increases the intensity of the tanning. As a result, it is possible to further reduce the concentration of the self-tanning substance. In addition, the hydrophilic solvents are able to ensure a more even distribution of the self-tanning substance, particularly when applied by misting.

The hydrophilic solvents to be used according to the invention can advantageously be chosen from the following groups of substances:
monoalcohols of low carbon number, e.g. isopropanol,
polyhydric alcohols, such as, preferably, propylene glycol or glycerol,
esters of fatty alcohols with alkanoic acids of low carbon number.

The hydrophilic solvents preferred according to the invention are propylene glycol and/or glycerol.

The preferred concentration of hydrophilic solvents, in particular propylene glycol and/or glycerol, in formulations according to the invention is in the range from 0.1 to 20 percent by weight.

In addition, the presence of so-called fatty carriers should lead to increased tanning intensity. The substances called fatty carriers according to the invention are generally also referred to as “sluices” since they transport the self-tanning agent molecules to deeper layers of the stratum corneum.

Fatty carriers to be mentioned here are, in particular, ceramides, cholesterol, phospholipids, cholesteryl sulphate, cholesteryl phosphate, phosphatidylcholine, lecithin and/or empty liposomes.

According to the invention, phospholipids means the following substances: phosphatidic acids, the actual lecithins, cardolipins, lysophospholipids, lysolecithins, plasmalogens, phosphosphingolipids, sphingomyelins. Preferred substances are described below.

Phosphatidic acids are glycerol derivatives which are esterified in the 1-sn and 2 position with fatty acids (1-sn position: mostly saturated, 2 position: mostly mono- or polyunsaturated), on atom 3-sn by contrast with phosphoric acid and characterized by the general structural formula

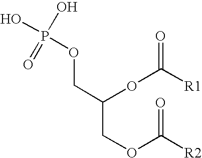

In the phosphatidic acids occurring in human or animal tissue, the phosphate radical is mostly esterified with amino alcohols, such as choline (lecithin=3-sn-phosphatidylcholine) or 2-aminoethanol (ethanolamine) or L-serine (cephalin=3-sn-phosphatidylethanolamine or sn-phosphatidyl-L-serine), with myo-inositol to give the phosphoinositides common in tissues [1-(3-sn-phosphatidyl)-d-myo-inositols], with glycerol to give phosphatidyl-glycerols. Particular preference is given to lecithins (=3-sn-phosphatidyl-choline).

Lecithins are characterized by the general structural formula 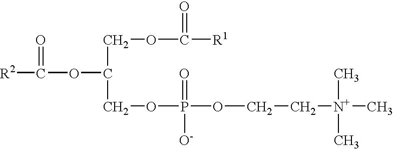
where R¹ and R² are typically unbranched aliphatic radicals having 15 or 17 carbon atoms and up to 4 cis double bonds.

Cardiolipins (1,3-bisphosphatidylglycerols) are phospholipids comprising two phosphatidic acids joined via glycerol.

Lysophospholipids are obtained when an acyl radical is cleaved off from phospholipids by phospholipase A (e.g. lysolecithins). Lysophospholipids are characterized by the general structural formula

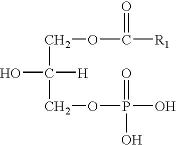

Lysolecithins, for example, are characterized by the general structural formula 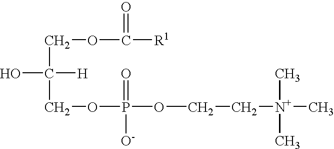
where R¹ is typically unbranched aliphatic radicals having 15 or 17 carbon atoms and up to 4 cis double bonds.

The phospholipids also include plasmalogens, in which instead of a fatty acid in the 1 position, an aldehyde (in the form of an enol ether) is bonded; the O-1-sn-alkenyl compounds corresponding to the phosphatidylcholines are, for example, called phosphatidalcholines.

As basic structure, the phosphosphingolipids are based on sphingosine or else phytosphingosine, which are characterized by the following structural formulae:

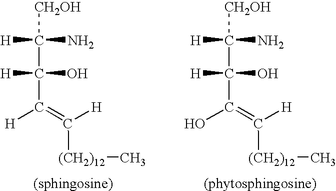

Modifications of sphingolipids are characterized, for example, by the general basic structure 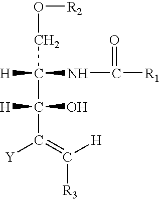
in which R₁ and R₃, independently of one another, are saturated or unsaturated, branched or unbranched alkyl radicals having 1 to 28 carbon atoms, R₂ is chosen from the group: hydrogen atom, saturated or unsaturated, branched or unbranched alkyl radicals having 1 to 28 carbon atoms, sugar radicals, phosphate groups which are unesterified or esterified with organic radicals, sulphate groups which are unesterified or esterified with organic radicals, and Y is either a hydrogen atom, a hydroxyl group or another heterofunctional radical.

Sphingophospholipids

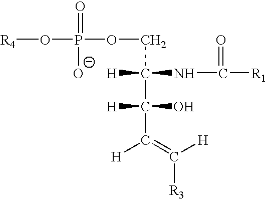

R₁ and R₃ are alkyl radicals, R₄ is an organyl radical. Sphingomyelins are organylphosphorylated sphingolipids of the type

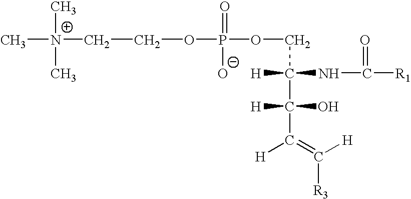

Particularly preferred phospholipids are lecithins. Lecithin types to be used advantageously are chosen from crude lecithins which have been deoiled and/or fractionated and/or spray-dried and/or acetylated and/or hydrolysed and/or hydrogenated. They are commercially available. Preference is given to soya lecithins.

According to the invention, use is advantageously made of ceramides, cholesterol, phospholipids, fatty acids, cholesteryl sulphate, cholesteryl phosphate, phosphatidylcholine, lecithin and/or empty liposomes.

Phospholipids to be used advantageously according to the invention can, for example, be acquired commercially under the trade names Phospholipon 25 or Phospholipon 90 (Natterman), Emulmetik 120 (Lucas Meyer), Sternpur E (Stern), Sternpur PM (Stern), Nathin 3KE (Stern), Phospholipon 90H (Nattermann/Rhone-Poulenc), Lipoid S 100 (Lipoid).

According to the invention, the preferred concentration of fatty carriers is in the range from 0.1 to 10 percent by weight.

It is furthermore preferred according to the invention when the formulations comprise UV filters. Since these UV filters also come into contact with the skin during application of the formulation, they should be UV filters which are compatible in the topical application. In this connection, an additional advantage which arises is that these UV filters likewise absorb evenly on the skin upon application and thus protect the skin against UV radiation.

Particular preference is given to those UV filters whose physiological safety has already been demonstrated. There are substances known from the specialist literature both for UV-A and also UV-B filters, e.g. benzylidene-camphor derivatives, such as 3-(4′-methylbenzylidene)-dl-camphor (e.g. Eusolex® 6300), 3-benzylidenecamphor (e.g. Mexoryl® SD), polymers of N-{(2 and 4)-[(2-oxoborn-3-ylidene)methyl]benzyl}acrylamide (e.g. Mexoryl® SW), N,N,N-trimethyl-4-(2-oxoborn-3-ylidenemethyl)anilinium methylsulphate (e.g. Mexoryl® SK) or (2-oxoborn-3-ylidene)toluene-4-sulphonic acid (e.g. Mexoryl® SL),
benzoyl- or dibenzoylmethanes, such as 1-(4-tert-butylphenyl)-3-(4-methoxyphenyl)propane-1,3-dione (e.g. Eusolex® 9020) or 4-isopropyl-dibenzoylmethane (e.g. Eusolex® 8020),
benzophenones, such as 2-hydroxy-4-methoxybenzophenone (e.g. Eusolex® 4360) or 2-hydroxy-4-methoxybenzophenone-5-sulphonic acid and its sodium salt (e.g. Uvinul® MS-40),
methoxycinnamic esters, such as octyl methoxycinnamate (e.g. Eusolex® 2292), isopentyl 4-methoxycinnamate, e.g. as a mixture of the isomers (e.g. Neo Heliopan® E 1000),
salicylate derivatives, such as 2-ethylhexyl salicylate (e.g. Eusolex® OS), 4-isopropylbenzyl salicylate (e.g. Megasol®) or 3,3,5-trimethylcyclohexyl salicylate (e.g. Eusolex® HMS),
4-aminobenzoic acid and derivatives, such as 4-aminobenzoic acid, 2-ethylhexyl 4-(dimethylamino)benzoate (e.g. Eusolex® 6007), ethoxylated ethyl 4-aminobenzoate (e.g. Uvinul® P25),
phenylbenzimidazolesulphonic acids, such as 2-phenylbenzimidazole-5-sulphonic acid and its potassium, sodium and triethanolamine salts (e.g. Eusolex® 232), 2,2-(1,4-phenylene)bisbenzimidazole-4,6-disulphonic acid and salts thereof (e.g. Neo Heliopan® AP) or 2,2-(1,4-phenylene)bis-benzimidazole-6-sulphonic acid;
and further substances, such as
2-ethylhexyl 2-cyano-3,3-diphenylacrylate (e.g. Eusolex® OCR),
3,3′-(1,4-phenylenedimethylene)bis(7,7-dimethyl-2-oxobicyclo[2.2.1]-hept-1-ylmethanesulphonic acid, and its salts (e.g. Mexoryl® SX) and
2,4,6-trianilino(p-carbo-2′-ethylhexyl-1′-oxy)-1,3,5-triazine (e.g. Uvinul® T 150)
hexyl 2-(4-diethylamino-2-hydroxybenzoyl)benzoate (e.g. Uvinul®UVA Plus, BASF).

The compounds listed are only to be regarded as examples. It is of course also possible to use other UV filters.

These organic UV filters are usually incorporated into cosmetic formulations in an amount of from 0.5 to 10 percent by weight, preferably 1-8%.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (e.g. Silatrizole®),
bis(2-ethylhexyl)4,4′-[(6-[4-((1,1-dimethylethyl)aminocarbonyl)phenyl-amino]-1,3,5-triazine-2,4-diyl)diimino]bis(benzoate) (e.g. Uvasorb® HEB),
dimethicone diethylbenzalmalonate (CAS No. 207 574-74-1)
2,2′-methylenebis-(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulphonic acid, mono-sodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).

Further suitable UV filters are also methoxyflavones corresponding to the earlier German patent application DE 10232595.2.

Organic UV filters are usually incorporated into cosmetic formulations in an amount of from 0.5 to 20 percent by weight, preferably 1-15%.

Conceivable inorganic UV filters are those from the group of titanium dioxides, such as, for example, coated titanium dioxide (e.g. Eusolex® T-2000, Eusolex®T-AQUA), zinc oxides (e.g. Sachtotec®), iron oxides and also cerium oxides. These inorganic UV filters are usually incorporated into cosmetic preparations in an amount of from 0.5 to 20 percent by weight, preferably 2-10%.

Preferred compounds with UV-filtering properties are 3-(4′-methyl-benzylidene)-dl-camphor, 1-(4-tert-butylphenyl)-3-(4-methoxyphenyl)-propane-1,3-dione, 4-isopropyldibenzoylmethane, 2-hydroxy-4-methoxy-benzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazol-5-sulphonic acid, and its potassium, sodium and triethanolamine salts.

Optimized compositions can, for example, comprise the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broadband protection, which can be further enhanced by adding inorganic UV filters, such as titanium dioxide microparticles.

All said UV filters can also be used in encapsulated form. In particular, it is advantageous to use organic UV filters in encapsulated form. Specifically, the following advantages arise:
The hydrophilicity of the capsule wall may be adjusted independently of the solubility of the UV filter. Thus, for example, even hydrophobic UV filters can be incorporated into purely aqueous preparations. In addition, the oily impression, often perceived as being unpleasant, upon application of the preparation comprising hydrophobic UV filters is suppressed.
Certain UV filters, in particular dibenzoylmethane derivatives, exhibit only reduced photostability in cosmetic preparations. By encapsulating these filters or compounds which impair the photostability of these filters, such as, for example, cinnamic acid derivatives, it is possible to increase the photostability of the entire preparation.
The literature discusses time and again the penetration of the skin by organic UV filters and the associated irritancy potential upon direct application to the human skin. The encapsulation of the corresponding substances that is proposed here suppresses this effect.
In general, by encapsulating individual UV filters or other ingredients it is possible to avoid preparation problems which arise as a result of individual preparation constituents interacting with one another, such as crystallization operations, precipitations and agglomeration, since the interaction is suppressed.

It is therefore preferred according to the invention when one or more of the abovementioned UV filters are present in encapsulated form. In this connection, it is advantageous if the capsules are so small that they cannot be observed with the naked eye. To achieve the abovementioned effects, it is further necessary that the capsules are sufficiently stable and do not release the encapsulated active ingredient (UV filter), or release it only to a low degree, into the surrounding area.

Suitable capsules can have walls made of inorganic or organic polymers. For example, U.S. Pat. No. 6,242,099 B1 describes the preparation of suitable capsules with walls made of chitin, chitin derivatives or polyhydroxylated polyamines. Capsules which are to be used particularly preferably according to the invention have walls which can be obtained by a sol gel process, as is described in the applications WO 00/09652, WO 00/72806 and WO 00/71084. Preference is given here in turn to capsules whose walls are made of silica gel (silica; undefined silicon oxide hydroxide). The preparation of the corresponding capsules is known to the person skilled in the art, for example from the cited patent applications, the contents of which also expressly belong to the subject-matter of the present application.

In this connection, the capsules are preferably present in preparations according to the invention in amounts which ensure that the encapsulated UV filters are present in the preparation in the amounts given above.

The preparations according to the invention can, moreover, comprise further customary gentle or skincare active ingredients. These may in principle be all active ingredients known to the person skilled in the art.

These may be chromone derivatives. In this connection, the term chromone derivative is preferably understood as meaning certain chromen-2-one derivatives which are suitable as active ingredients for the preventative treatment of human skin and human hair against ageing processes and harmful environmental influences. At the same time, they display a low irritation potential for the skin, have a positive influence on the water binding in the skin, maintain or increase the elasticity of the skin and thus promote skin smoothing. These compounds preferably correspond to the formula II 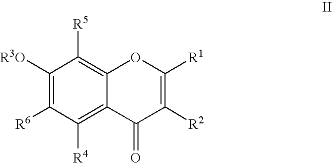
where
R¹ and R² may be identical or different and are chosen from
   H, —C(═O)—R⁷, —C(═O)—OR⁷,
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and in addition the alkyl chain may also be interrupted by oxygen, and/or
   C₃- to C₁₀-cycloalkyl groups and/or C₃- to C₁₂-cycloalkenyl groups, where the rings may in each case also be bridged by —(CH₂)ₙ groups where
n=1 to 3,
R³ is H or straight-chain or branched C₁- to C₂₀-alkyl groups,
R⁴ is H or OR⁸,
R⁵ and R⁶ may be identical or different and are chosen from
   −H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and in addition the alkyl chain may also be interrupted by oxygen and
R⁷ is H, straight-chain or branched C₁- to C₂₀-alkyl groups, a polyhydroxy compound, such as preferably an ascorbic acid radical or glycosidic radicals and
R⁸ is H or straight-chain or branched C₁- to C₂₀-alkyl groups, where at least 2 of the substituents R¹, R², R⁴-R⁶ are different from H, or at least one substituent of R¹ and R² is —C(═O)—R⁷ or —C(═O)—OR⁷.

The proportion of one or more compounds chosen from chromone derivatives in the preparation according to the invention is preferably from 0.001 to 5 percent by weight, particularly preferably from 0.01 to 2 percent by weight, based on the total preparation.

A protective effect against oxidative stress or against the effect of free radicals of the formulations according to the invention can be achieved when the preparations comprise one or more antioxidants, the person skilled in the art being presented with no difficulties at all in selecting antioxidants which act suitably rapidly or in a time-delayed manner.

There are many proven substances known from the specialist literature which can be used as antioxidants, e.g. amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserin), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulphoximine compounds (e.g. buthionine sulphoximines, homocysteine sulphoximine, buthionine sulphones, penta-, hexa-, heptathionine sulphoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extract, bilirubin, biliverdin, EDTA, EGTA and derivatives thereof, unsaturated fatty acids and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, tri-hydroxybutyrophenone, quercitin, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid (e.g. Oxynex® AP), natural tocopherols, L-(+)-ascorbyl palmitate, L-(+)-ascorbic acid and citric acid (e.g. Oxynex® K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butylhydroxytoluene (BHT), L-(+)-ascorbyl palmitate and citric acid (e.g. Oxynex® 2004). Antioxidants of this type are used with compounds of the formula I in such compositions usually in ratios in the range from 1000:1 to 1:1000, preferably in amounts of from 100:1 to 1:100.

The preparations according to the invention may comprise vitamins as further ingredients. Preferably, vitamins and vitamin derivatives chosen from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamin (vitamin B₁₂) are present in the cosmetic preparations according to the invention, particularly preferably vitamin A palmitate, vitamin C and derivatives thereof, DL-α-tocopherol, tocopherol E acetate, nicotinic acid, pantothenic acid and biotin. Vitamins are used here with compounds of the formula I usually in ratios in the range from 1000:1 to 1:1000, preferably in amounts of from 100:1 to 1:100.

Among the phenols with an antioxidative effect, the polyphenols, some of which occur as natural substances, are particularly interesting for applications in the pharmaceutical, cosmetic or nutrition field. For example, the flavonoids or bioflavonoids, known primarily as plant dyes, often have an antioxidative potential. Effects of the substitution pattern of mono- and dihydroxyflavones are dealt with by K. Lemanska, H. Szymusiak, B. Tyrakowska, R. Zielinski, I. M. C. M. Rietjens; Current Topics in Biophysics 2000, 24(2), 101-108. It is observed therein that dihydroxyflavones with an OH group adjacent to the keto function or OH groups in 3′4′ or 6, 7 or 7,8 position have antioxidative properties, whereas some other mono- and dihydroxyflavones have no antioxidative properties.

Quercetin (cyanidanol, cyanidenolon 1522, meletin, sophoretin, ericin, 3,3′,4′,5,7-pentahydroxyflavone) is often specified as a particularly effective antioxidant (e.g. C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881 investigate the pH dependency of the antioxidative effect of hydoxyflavones. Over the entire pH range, quercetin exhibits the highest activity of the investigated structures.

Suitable antioxidants are also compounds of the formula III 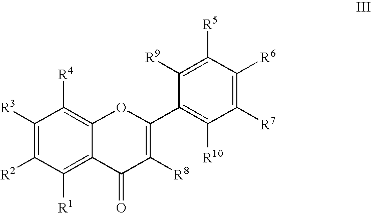
where R¹ to R¹⁰ may be identical or different and are chosen from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and in addition the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ groups where n=1 to 3,
where all OR¹¹, independently of one another, are
   OH
   straight-chain or branched C₁- to C₂₀-alkyloxy groups,
   straight-chain or branched C₃- to C₂₀-alkenyloxy groups,
   straight-chain or branched C₁- to C₂₀-hydroxyalkoxy groups, where the hydroxyl group(s) may be bonded to a primary or secondary carbon atom of the chain and in addition the alkyl chain may also be interrupted by oxygen, and/or
   C₃- to C₁₀-cycloalkyloxy groups and/or C₃- to C₁₂-cycloalkenyloxy groups, where the rings may in each case also be bridged by —(CH₂)ₙ groups where n=1 to 3 and/or
   mono- and/or oligoglycosyl radicals,
with the proviso that at least 4 radicals from R¹ to R⁷ are OH and that at least 2 pairs of adjacent —OH groups are present in the molecule,
or R², R⁵ and R⁶ are OH and the radicals R¹, R³, R⁴ and R⁷⁻¹⁰ are H, as are described in the earlier German patent application DE 10244282.

Particularly preferred active ingredients are also pyrimidinecarboxylic acids and/or aryl oximes.

Pyrimidinecarboxylic acids occur in halophilic microorganisms and play a role in the osmoregulation of these organisms (E. A. Galinski et al., Eur. J. Biochem., 149 (1985) page 135-139). In this connection, among the pyrimidinecarboxylic acids, mention is made in particular of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid and derivatives thereof. These compounds stabilize enzymes and other biomolecules in aqueous solutions and organic solvents. In addition, they stabilize in particular enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be used for the preparation of a medicament for the treatment of skin disorders. Other fields of use of hydroxyectoin and other ectoin derivatives are typically in fields in which, for example, trehalose is used as additive. Thus, ectoin derivatives, such as hydroxyectoin, can be used as protectant in dried yeast and bacteria cells. Pharmaceutical products such as non-glycosylated, pharmaceutically active peptides and proteins, e.g. t-PA, can also be protected with ectoin or its derivatives.

Among the cosmetic applications, mention is made in particular of the use of ectoin and ectoin derivatives for the care of aged, dry or irritated skin. For example, European patent application EP-A-0 671 161 describes, in particular, that ectoin and hydroxyectoin are used in cosmetic preparations such as powders, soaps, surfactant containing cleansing products, lipsticks, blusher, foundations, care creams and sunscreen preparations.

In this connection, preference is given to using a pyrimidinecarboxylic acid according to formula IV below, 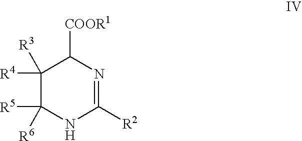
in which R¹ is a radical H or C₁₋₈-alkyl, R² is a radical H or C₁₋₄-alkyl and R³, R⁴, R⁵ and R⁶ are in each case independently of one another a radical from the group H, OH, NH₂ and C₁₋₄-alkyl. Preference is given to using pyrimidinecarboxylic acids in which R² is a methyl or an ethyl group, and R¹ or R⁵ and R⁶ are H. Particular preference is given to using the pyrimidine-carboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). In this connection, the preparations according to the invention comprise pyrimidinecarboxylic acids of this type preferably in amounts up to 15% by weight.

Among the aryl oximes, preference is given to using 2-hydroxy-5-methyllaurophenone oxime, which is also referred to as HMLO, LPO or F5. Its suitability for use in cosmetic compositions is known, for example, from German laid-open specification DE-A-41 16 123. Preparations which comprise 2-hydroxy-5-methyllaurophenone oxime are accordingly suitable for the treatment of skin disorders which are accompanied by inflammations. It is known that preparations of this type can be used, for example, for the therapy of psoriasis, various forms of eczema, irritative and toxic dermatitis, UV dermatitis, and other allergic and/or inflammatory disorders of the skin and of skin appendages. In this connection, the preparations preferably comprise 0.01 to 10% by weight of the aryl oxime, it being particularly preferred if the preparation comprises 0.05 to 5% by weight of aryl oxime.

In addition, the preparations according to the invention can also comprise dyes and colour pigments. The dyes and colour pigments can be chosen from the corresponding positive list of the Cosmetics Directive or the EC list of cosmetic colorants. In most cases, they are identical to the dyes permitted for foods. Advantageous colour pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or colour pigments from the following list. The Colour Index numbers (CIN) are taken from the Rowe Colour Index, 3rd edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulphonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenene | 11920 | Orange |
| Solvent Red 3 | 12010 | Red |
| 1-(2′-Chloro-4′-nitro-1′-phenylazo)-2-hydroxynaphthalene | 12085 | Red |
| Pigment Red 3 | 12120 | Red |
| Ceres red; Sudan red; Fat Red G | 12150 | Red |
| Pigment Red 112 | 12370 | Red |
| Pigment Red 7 | 12420 | Red |
| Pigment Brown 1 | 12480 | brown |
| 4-(2′-Methoxy-5′-sulphodiethylamido-1′-phenylazo)-3- | 12490 | red |
| hydroxy-5″-chloro-2″,4″-dimethoxy-2-naphthanilide | | |
| Disperse Yellow 16 | 12700 | yellow |
| 1-(4-Sulpho-1-phenylazo)-4-aminobenzene-5-sulphonic | 13015 | yellow |
| acid | | |
| 2,4-Dihydroxyazobenzene-4′-sulphonic acid | 14270 | orange |
| 2-(2,4-Dimethylphenylazo-5-sulpho)-1-hydroxynaphthalene- | 14700 | Red |
| 4-sulphonic acid | | |
| 2-(4-Sulpho-1-naphthylazo)-1-naphthol-4-sulphonic acid | 14720 | Red |
| 2-(6-Sulpho-2,4-xylylazo)-1-naphthol-5-sulphonic acid | 14815 | Red |
| 1-(4′-Sulphophenylazo)-2-hydroxynaphthalene | 15510 | orange |
| 1-(2-Sulpho-4-chloro-5-carboxy-1-phenylazo)-2- | 15525 | Red |
| hydroxynaphthalene | | |
| 1-(3-Methylphenylazo-4-sulpho)-2-hydroxynaphthalene | 15580 | Red |
| 1-(4′,(8′)-Sulphonaphthylazo)-2-hydroxynaphthalene | 15620 | Red |
| 2-Hydroxy-1,2′-azonaphthalene-1′-sulphonic acid | 15630 | Red |
| 3-Hydroxy-4-phenylazo-2-naphthylcarboxylic acid | 15800 | Red |
| 1-(2-Sulpho-4-methyl-1-phenylazo)-2- | 15850 | Red |
| naphthylcarboxylic acid | | |
| 1-(2-Sulpho-4-methyl-5-chloro-1-phenylazo)-2-hydroxynaphthalene- | 15865 | Red |
| 3-carboxylic acid | | |
| 1-(2-Sulpho-1-naphthylazo)-2-hydroxynaphthalene-3- | 15880 | Red |
| carboxylic acid | | |
| 1-(3-Sulpho-1-phenylazo)-2-naphthol-6-sulphonic acid | 15980 | orange |
| 1-(4-Sulpho-1-phenylazo)-2-naphthol-6-sulphonic acid | 15985 | yellow |
| Allura Red | 16035 | red |
| 1-(4-Sulpho-1-naphthylazo)-2-naphthol-3,6-disulphonic | 16185 | red |
| acid | | |
| Acid Orange 10 | 16230 | orange |
| 1-(4-Sulpho-1-naphthylazo)-2-naphthol-6,8-disulphonic | 16255 | red |
| acid | | |
| 1-(4-Sulpho-1-naphthylazo)-2-naphthol-3,6,8- | 16290 | red |
| trisulphonic acid | | |
| 8-Amino-2-phenylazo-1-naphthol-3,6-disulphonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulpho-1-phenylazo)-1-(4-sulphophenyl)-5- | 19140 | yellow |
| hydroxy-pyrazolone-3-carboxylic acid | | |
| Pigment Yellow 16 | 20040 | yellow |
| 2,6-(4′-Sulpho-2″,4″-dimethyl)bisphenylazo)1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulpho-1″-phenylazo)-7′-sulpho-1′- | 27755 | black |
| naphthylazo]-1-hydroxy-7-aminonaphthalene-3,6- | | |
| disulphonic acid | | |
| 4-[4″-Sulpho-1″-phenylazo)-7′-sulpho-1′-naphthylazo]-1- | 28440 | black |
| hydroxy-8-acetylaminonaphthalene-3,5-disulphonic acid | | |
| Direct Orange 34, 39, 44, 46, 60 | 40215 | orange |
| Food Yellow | 40800 | orange |
| trans-β-Apo-8′-carotinaldehyde (C₃₀) | 40820 | orange |
| trans-Apo-8′-carotinic acid (C₃₀)-ethyl ester | 40850 | orange |
| Canthaxanthin | 40850 | orange |
| Acid Blue 1 | 42045 | blue |
| 2,4-Disulpho-5-hydroxy-4′-4″-bis-(diethylamino-) | 42051 | blue |
| triphenylcarbinol | | |
| 4-[(-4-N-Ethyl-p-sulphobenzylamino)phenyl-(4-hydroxy- | 42053 | green |
| 2-sulphophenyl)(methylene)-1-(N-ethyl-N-p-sulphobenzyl)- | | |
| 2,5-cyclohexadienimine] | | |
| Acid Blue 7 | 42080 | blue |
| (N-Ethyl-p-sulphobenzylamino)phenyl-(2-sulphophenyl)- | 42090 | blue |
| methylene-(N-ethyl-N-p-sulphobenzyl)Δ^{2,5}-cyclohexadienimine | | |
| Acid Green 9 | 42100 | green |
| Diethyl-di-sulphobenzyldi-4-amino-2-chlorodi-2-methylfuchsonimmonium | 42170 | green |
| Basic Violet 14 | 42510 | violet |
| Basic Violet 2 | 42520 | violet |
| 2′-Methyl-4′-(N-ethyl-N-m-sulphobenzyl)amino-4″-(N- | 42735 | blue |
| diethyl)amino-2-methyl-N-ethyl-N-m-Sulphobenzyl- | | |
| fuchsonimmonium | | |
| 4′-(N-Dimethyl)amino-4″-(N-phenyl)aminonaphtho-N- | 44045 | blue |
| dimethylfuchsonimmonium | | |
| 2-Hydroxy-3,6-disulpho-4,4′-bisdimethylaminonaphtho- | 44090 | green |
| fuchsonimmonium | | |
| Acid Red 52 | 45100 | red |
| 3-(2′-Methylphenylamino)-6-(2′-methyl-4′-sulphophenyl- | 45190 | violet |
| amino)-9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulphonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminium complex | 58000 | red |
| 3-Oxypyrene-5,8,10-sulphonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulpho-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo-disulphonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6, 19; Natural Red 1 | 75100 | yellow |
| Bixin, Norbixin | 75120 | orange |
| Lycopene | 75125 | yellow |
| trans-alpha-, beta- and gamma-carotene | 75130 | orange |
| Keto- and/or hydroxyl derivatives of carotene | 75135 | yellow |
| Guanine or pearlescent agent | 75170 | white |
| 1,7-Bis(4-hydroxy-3-methoxyphenyl)-1,6-heptadiene- | 75300 | yellow |
| 3,5-dione | | |
| Complex salt (Na, Al, Ca) of carminic acid | 75470 | red |
| Chlorophyll a und b; copper compounds of chlorophylls | 75810 | green |
| and chlorophyllins | | |
| Aluminium | 77000 | white |
| Hydrated alumina | 77002 | white |
| Hydrous aluminium silicate | 77004 | white |
| Ultramarine | 77007 | blue |
| Pigment Red 101 and 102 | 77015 | red |
| Barium sulphate | 77120 | white |
| Bismuth oxychloride and its mixtures with mica | 77163 | white |
| Calcium carbonate | 77220 | white |
| Calcium sulphate | 77231 | white |
| Carbon | 77266 | black |
| Pigment Black 9 | 77267 | black |
| Carbo medicinalis vegetabilis | 77268 | black |
| Chromium oxide | 77288 | green |
| Chromium oxide, hydrous | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II)- and iron(III)hexacyanoferrate | 77510 | blue |
| Pigment White 18 | 77713 | white |
| Manganese ammonium diphosphate | 77742 | violet |
| Manganese phosphate; Mn₃(PO₄)₂.7 H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavine | | yellow |
| Sugar colouring | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol blue | | blue |

It may also be favourable to choose as dye one or more substances from the following group: 2,4-dihydroxyazobenzene, 1-(2′-chloro-4′-nitro-1′-phenylazo)-2-hydroxynaphthalene, Ceres Red, 2-(4-sulpho-1-naphthylazo)-1-naphthol-4-sulphonic acid, calcium salt of 2-hydroxy-1,2′-azonaphthalene-1′-sulphonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminium salt of 1-(4-sulpho-1-phenylazo)-2-naphthol-6-sulphonic acid, aluminium salt of 1-(4-sulpho-1-naphthylazo)-2-naphthol-3,6-disulphonic acid, 1-(4-sulpho-1-naphthylazo)-2-naphthol-6,8-disulphonic acid, aluminium salt of 4-(4-sulpho-1-phenylazo)-1-(4-sulphophenyl)-5-hydroxypyrazolone-3-carboxylic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, aluminium salt of 2,4,5,7-tetraiodofluorescein, aluminium salt of quinophthalone disulphonic acid, aluminium salt of indigo disulphonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extract, β-carotene or cochineal.

Also advantageous for the purposes of the present invention are gel creams with a content of pearlescent pigments. Preference is given in particular to the types of pearlescent pigments listed below:
1. Natural pearlescent pigments, such as, for example
   1. “pearl essence” (guanine/hypoxanthin mixed crystals from fish scales) and
   2. “mother of pearl” (ground mussel shells)
2. Monocrystalline pearlescent pigments, such as, for example, bismuth oxychloride (BiOCl)
3. Layer-substrate pigments: e.g. mica/metal oxide.

Bases for pearlescent pigments are, for example, pulverulent pigments or castor oil dispersions of bismuth oxychloride and/or titanium dioxide, and bismuth oxychloride and/or titanium dioxide on mica. The lustre pigment listed under CIN 77163, for example, is particularly advantageous.

Also advantageous are, for example, the following types of pearlescent pigment based on mica/metal oxide:

| Group | Coating/layer thickness | Colour |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Colour lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |

Particular preference is given, for example, to the pearlescent pigments obtainable from Merck under the trade names Timiron, Colorona or Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines, are advantageous.

It can moreover be advantageous to dispense completely with a substrate such as mica. Particular preference is given to pearlescent pigments which are prepared using SiO₂. Such pigments, which may also additionally have goniochromatic effects, are available, for example, under the trade name Sicopearl Fantastico from BASF.

In addition, pigments from Engelhard/Mearl based on calcium sodium borosilicate which have been coated with titanium dioxide can advantageously be used. These are available under the name Reflecks. In addition to the colour, they have a glitter effect as a result of their particle size of 40-80 μm.

In addition, also particularly advantageous are effect pigments which are obtainable under the trade name Metasomes Standard/Glitter in various colours (yellow, red, green, blue) from Flora Tech. The glitter particles are present here in mixtures with various auxiliaries and dyes (such as, for example, the dyes with the Colour Index (CI) Numbers 19140, 77007, 77289, 77491).

The dyes and pigments may be present either individually or in a mixture, and can be mutually coated with one another, different coating thicknesses generally giving rise to different colour effects. The total amount of dyes and colour-imparting pigments is advantageously chosen from the range from e.g. 0.1% by weight to 30% by weight, preferably from 0.5 to 15% by weight, in particular from 1.0 to 10% by weight, in each case based on the total weight of the preparations.

All compounds or components which can be used in the preparations are either known and available commercially or can be synthesized by known processes.

The preparations according to the invention can, moreover, comprise further customary skin-friendly or skincare active ingredients. These may in principle be all active ingredients known to the person skilled in the art.

The composition of the present invention may be in the form of liquid, creamy, milky or gel-like bath additives which are added as liquid together with the bath water, or in bath capsules which preferably consist of gelatin and which dissolve in the bathwater and release the composition of the present invention.

The present invention thus further provides a composition comprising at least one self-tanning substance, characterized in that the formulation is liquid, creamy, milky and/or gel-like bath additives, bath tablets, bath salts and/or bath capsules.

One possible composition of the liquid formulation comprises up to 75% surfactants (anionic, cationic, nonionic or amphoteric), up to 10% viscosity agents, such as fatty alcohols, up to 5% combability and conditioning agents, up to 5% further ingredients, such as refatting agents, thickeners, opacifiers or pigments, up to 5% perfume oils, up to 1% preservatives, up to 0.5% sequestrants, up to 1% dyes, 0.1-1% DHA, UV filters, 0.1-20% propylene glycol and/or glycerol and 0.1 and 10% fatty carriers and is made up to 100% with water.

The composition of the present invention may also be present in bath additives such as bath tablets or bath salts. One possible composition of the solid formulation comprises up to 90% sodium salts (e.g. sodium carbonate, bicarbonate, sesquicarbonate, chloride, thiosulphate, borate, phosphate or citrate), up to 40% organic acids (e.g. tartaric acid, citric acid) for effervescent preparations, up to 5% perfume oils (essential oils), up to 5% skincare substances, up to 5% plant oils, up to 5% fillers and for tablets, disintegration auxiliaries (e.g. dextrin, silica, cellulose, gum), up to 5% binders, up to 2% surfactants, up to 1% dyes, 0.1-1% DHA, UV filters, 0.1-20% propylene glycol and/or glycerol and 0.1 and 10% fatty carriers.

In addition, it is preferred when the self-tanning formulations comprise moisture-donating substances, such as, for example, erythrulose or the abovementioned ectoins.

Surprisingly, it has now been found that the required active ingredient concentration of compounds according to formula I and additional tanning agents can be reduced if the self-tanning agents are applied at elevated temperature.

It is assumed that the improved tanning effect at elevated temperatures in the case of 1,3-dihydroxyacetone is connected with the following mechanism. As a raw material, DHA is in the form of a powder and consists of dimers. Dissolved in water, some of the dimers convert to the active monomeric form, which brings about the tanning reaction. At an elevated water temperature, the amount of monomers increases. For example, it has been found that in DHA solutions at 30-50° C. up to 30% more active DHA monomers are present than in DHA solutions at 20° C. At the same time, the elevated temperature increases the reaction rate of the tanning reaction.

The present invention thus provides the use of at least one self-tanning substance according to formula I or a formulation comprising at least one self-tanning substance according to formula I for application to the human skin, with application taking place at elevated temperature.

For this reason, it is preferred according to the invention if the application temperature is in the range between 25 and 60° C., preferably between 30 and 55° C. and particularly preferably between 37 and 50° C.

The present invention further provides a method of tanning the human body, which is characterized in that at least one self-tanning substance according to formula I or a formulation comprising at least one self-tanning substance according to formula I is dissolved in water, the solution is brought to a temperature which is elevated relative to room temperature and the solution is applied to the human body.

In addition, it has been found that the equilibrium of the monomer to dimer concentration is established within about 15 minutes following-dissolution. It is therefore preferred according to the invention when the solution of the self-tanning substance is heated for about 15 min, but at least about 10 min, before the solution is applied to the human skin.

In a particularly comfortable manner, said effect can be exploited when used in bath tubs.

The required evenness of tanning can only be achieved with difficulty, or not at all, by mere rubbing. In addition, some areas of the body, in particular on the back, can only be reached with difficulty during self-application of a cream. These problems are avoided with application as bath water. In addition, the application can take place during the customary bathing time, and penetration of the self-tanning agents into the deeper layers of horny skin is favoured by the softening of the skin during bathing.

According to the invention, it is therefore particularly preferred when the solution is applied in a bathtub or whirlpool. The intensive and long-lasting contact of the skin with the active ingredient solution additionally achieves particularly even tanning, which is, in addition, possible with particularly low active ingredient concentrations.

Whirlpools or other baths with an agitated surface in particular offer the additional advantage that no line arises in the neck area, but a continuous fading of the tan arises. If the intention is to also tan the face, then this can be done in a classical way by applying a self-tan-containing cream or by misting with a self-tan solution.

In the corresponding process according to the invention, the human body, as a whole or partially, is immersed into the solution.

Another method, preferred according to the invention, of applying self-tan solutions to the skin is misting, which can take place, for example, by means of a shower or spray gun.

In the corresponding process, for even tanning, the human body—completely or partially—is sprayed evenly with the heated solution.

The skin tanning achieved in this way cannot be washed off and is removed only with normal shedding of the skin (after about 10-15 days).

Particularly in the case of application as a bath, it may be further preferred for a water-repelling preparation to be applied to parts of the body which are not to be tanned, or are to be tanned only slightly. Such preparations can be prepared on the basis of silicones, paraffins, various organic polymers, petroleum or fatty acid salts, such as stearates. During bathing, they prevent or reduce the treated skin coming into contact with the self-tanning agent and thus the achieved tanning. Particularly on parts of the body with thickened horny skin, such a pretreatment may be advisable in order to prevent intense coloration of these areas.

A further subject of the invention relates to the use of these cosmetic or dermatological compositions for the purpose of artificially coloring the skin.

An additional subject of the invention relates to a process for the artificial coloring of the skin using, by application, a composition as described above in the text.

Having generally described this invention, a further understanding can be obtained by reference to certain specific examples which are provided herein for purposes of illustration only and are not intended to be limiting unless otherwise specified.

Examples of compositions according to the invention will now be given in a non-limiting manner.

Although the present invention has been described with reference to specific details of certain embodiments thereof, it is not intended that such details should be regarded as limitations upon the scope of the invention except insofar as they are included in the accompanying claims.

### EXAMPLES

### Example 1

### Synthesis of 2,5-dipalmitoyloxymethyl-[1,4]dioxane-2,5-diol

Method A

To a solution of DHA (40.0 g, 0.44 mol) in dichloromethane (330 mL) and pyridine (130 mL) at 0° C. under nitrogen atmosphere is added palmitoyl chloride (14.4 mL, 0.05 mol), and the reaction mixture is stirred at room temperature for 24 h. The reaction is quenched by slow addition of an aqueous solution of 1 N HCl, and the resulting mixture partitioned over 1 N HCl (250 mL) and dichloromethane (250 mL). The organic phase is washed with saturated sodium bicarbonate, dried over MgSO₄, and concentrated in vacuo. The resulting solid residue is carefully recrystallized from toluene to afford 8.3 g of the 2,5-dipalmitoyloxymethyl-[1,4]dioxane-2,5-diol as white needles. The monomer form, present in the filtrate, slowly reverts to the dimer 2,5-dipalmitoyloxymethyl-[1,4]dioxane-2,5-diol when allowed to stand at room temperature.

Method B

Palmitic acid (5.70 g, 22.2 mmol) and DMAP (2.71 g, 22.2 mmol) are added into a stirred solution of DHA (1.00 g, 11.1 mmol) in CH₂Cl₂ (200 ml) in a 500 ml 2-necked round bottom flask at room temperature under nitrogen atmosphere. A solution of DCC (4.59 g, 22.2 mmol) in CH₂Cl₂ (20 ml) is added dropwise into the reaction mixture. After stirring for 20 hrs at room temperature, the precipitated dicyclohexylurea is removed by filtration. The dicyclohexylurea is washed with excess of CH₂Cl₂ to remove the product that might remain. Then the filtrate is washed with a 5% aqueous solution of HCl (100 ml), followed by distilled H₂O (200 ml) and saturated NaCl solution (150 ml). The organic layer is then dried with sodium sulfate and evaporated under reduce pressure. After the evaporation, the residue is recrystallized from EtOAc/petrolether to give colorless crystals of 2,5-dipalmitoyloxymethyl-[1,4]dioxane-2,5-diol.

m.p.: 95.2° C.

¹H NMR (500 MHz, CDCl₃) δ: 4.23 (d, 2H, J=11.8 Hz), 4.08 (d, 2H, J=11.8 Hz), 4.00 (d, 2H, J=11.8 Hz), 3.60 (d, 2H, J=11.8 Hz), 3.10 (s, 2H), 2.37 (t, 4H, J=7.5 Hz), 1.60-1.66 (m, 4H), 1.26-1.29 (m, 48H), 0.88 (t, 6H, J=6.8 Hz).

HPLC-APCI-MS: 674 (M++NH₄+)

Using analogous reactions analogous dimeric DHA esters with different alkyl chain length such as 2-oxopropyl 1,3-didodecanoate or 2-oxopropyl 1,3-ditetradecanoate can be obtained.

### Example 2

### Foam Bath

| Ingredient | [%] |
|---|---|
| 2,5-dipalmitoyloxymethyl- | 0.1-1 |
| [1,4]dioxane-2,5-diol | |
| Surfactant | 10-20 |
| Phospholipids | 5 |
| Preservative | q.s. |
| Colorant | q.s. |
| Perfume oil | q.s. |
| Water | ad 100 |

Preparation:
The ingredients are mixed.

### Example 3

### Formulation for Misting in Active-Ingredient Showers

| Ingredient | [%] |
|---|---|
| 2,5-dipalmitoyloxymethyl- | 5 |
| [1,4]dioxane-2,5-diol | |
| Propylene glycol | 10 |
| Phospholipids | 5 |
| Preservative | q.s. |
| Perfume oil | q.s. |
| Water | ad 100 |

Preparation:
The ingredients are mixed.

### Example 4

### Self-Tanning Suncream

A. Emulsion containing the dihydroxyacetone ester 2,5-
dipalmitoyloxymethyl-[1,4]dioxane-2,5-diol:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| 2,5-dipalmitoyloxymethyl-[1,4]dioxane-2,5-diol | 14.6% |
| Aqueous phase: | |
| Phenoxyethanol (preserving agent) | 0.5% |
| Water | qs 100% |

B. Emulsion containing lipase:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| Aqueous phase: | |
| Phenoxyethanol (preserving agent) | 0.5% |
| Lipase SP644 | 2% |
| Water | qs 100% |

Emulsions A and B are placed in two separate compartments and mixed together at the moment of application to the skin.

After application to the skin, the product obtained gives the skin a progressively tanned coloration.

### Example 5

### Self-Tanning Suncream

A. Emulsion containing 2,5-dipalmitoyloxymethyl-
[1,4]dioxane-2,5-diol:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| 2,5-dipalmitoyloxymethyl-[1,4]dioxane-2,5-diol | 10% |
| Aqueous phase: | |
| Phenoxyethanol (preserving agent) | 0.5% |
| Water | qs 100% |

B. Emulsion containing lipase:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| Aqueous phase: | |
| Phenoxyethanol (preserving agent) | 0.5% |
| Lipase 100 L | 1% |
| Water | qs 100% |

The emulsions are placed in two different compartments and are brought into contact at the moment of application.

### Example 6

### Self-Tanning Suncream

A. Emulsion containing 2,5-dipalmitoyloxymethyl-
[1,4]dioxane-2,5-diol:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| 2,5-dipalmitoyloxymethyl-[1,4]dioxane-2,5-diol | 10% |
| Aqueous phase: | |
| Phenoxyethanol (preserving agent) | 0.5% |
| Water | qs 100% |

B. Emulsion containing lysine:

| | |
|---|---|
| Oily phase: | |
| Steareth-2 (surfactant) | 3% |
| Steareth-21 (surfactant) | 2% |
| PPG-15 stearyl ether (surfactant) | 29.5% |
| Aqueous phase: | |
| Phenoxyethanol (preserving agent) | 0.5% |
| Lysine | 5% |
| Water | qs 100% |

Emulsions A and B are placed in two separate compartments and mixed together at the moment of application to the skin.

After application to the skin, the product obtained gives the skin a progressively tanned coloration.

### Example 7

### Maillard Browning Test

The ability of compounds according to formula I to induce an artificial tanning on the skin can roughly be estimated using a simple Maillard browning test. This visual test is based on the principle that the reaction of skin with DHA to produce an artificial tan proceeds through a complex set of reactions (Maillard reaction) with free amino acids of skin proteins, particularly with arguinine, lysine and histidine.

The tanning ability of 2,5-dipalmitoyloxymethyl-[1,4]dioxane-2,5-diol (A) is evaluated by means of the Maillard browning effect after reacting with lysine in different solvents (water and the cosmetic oil miglyol) and compared with those of reference compounds: DHA and monomeric DHA-palmitic acid monoester (B) under the same conditions. Besides that, the tanning effect of 2,5-dipalmitoyloxymethyl-[1,4]dioxane-2,5-diol in the presence of lipases (enzymes used by the body to hydrolyse fats) is also studied.

| Time | | A | B | DHA |
|---|---|---|---|---|
| 20 | min | — | — | — |
| 1 | h | — | Light yellow | — |
| 5 | h | Light yellow | Yellow | Light yellow |
| 1 | day | Yellow | Brown | Brown |
| 3 | days | ″ | ″ | ″ |
| 8 | days | Brown | Dark brown | Dark brown |

Tanning test 1. - 1 mL of a 0.1M solution or suspension of the test substance in miglyol is allowed to react at room temperature with 1 mL of a 1M Lysine aqueous solution.

| | | | A + 1% | | B + 1% | |
|---|---|---|---|---|---|---|
| Time | | A | lipase | B | lipase | DHA |
| 20 | min | — | — | — | — | Light |
| | | | | | | yellow |
| 1 | h | — | — | Light | Yellow | Brown |
| | | | | yellow | | |
| 5 | h | — | Light | Yellow | Dark | Dark |
| | | | yellow | | yellow | brown |
| 1 | day | Light | Dark | Brown | Brown | Dark |
| | | yellow | yellow | | | brown |
| 2 | days | Brown | Dark | Dark | Dark | Dark |
| | | | brown | brown | brown | brown |

Tanning test 2. - 1 mL of a 0.1M aqueous solution or suspension of the test substance is allowed to react at room temperature with 1 mL of a 1M Lysine aqueous solution.

The results show that compound A is able to undergo Maillard browning reactions. The presence of lipases favours the appearance of the artificial tan.

1. A cosmetic or dermatological composition comprising, in a cosmetically or dermatologically acceptable vehicle, at least one esterified dihydroxyacetone derivative corresponding to the general formula I 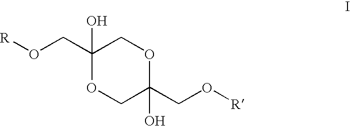
wherein R and R′ represent a hydrogen atom or a saturated or unsaturated, linear, branched or cyclic, optionally hydroxylated acyl radical having from 2 to 25 carbon atoms, and where R and R′ are identical or different on condition that they are never simultaneously a hydrogen atom.

2. A composition according to claim 1, further containing at least one compound capable of cleaving at least one ester bond.

3. Composition according to claim 1, in which said acyl radical is a benzoyl radical, an alkylbenzoyl radical, an acylbenzoyl radical or a 2-hydroxy-2-phenylacetyl radical.

4. Composition according to claim 1, in which said acyl radical is hydroxylated.

5. Composition according to claim 1, in which said acyl radical has from 3 to 18 carbon atoms.

6. Composition according to claim 1, in which said esterified derivative is selected from the group consisting of 2-oxopropyl 1,3-didodecanoate, 2-oxopropyl 1,3-dihexadecanoate and 2-oxopropyl 3-hexadecanoate.

7. Composition according to claim 1, in which said esterified derivative is at a concentration ranging from 0.1% to 20% by weight relative to the total weight of the composition.

8. Composition according to claim 1, in which said esterified derivative is at a concentration ranging from 0.5% to 10% by weight relative to the total weight of the composition.

9. Composition according to claim 2, in which said compound capable of cleaving at least one ester bond is a nucleophilic compound.

10. Composition according to claim 2, in which said compound capable of cleaving at least one ester bond is an amine, an anion, or an enzyme.

11. Composition according to claim 2, in which said compound capable of cleaving at least one ester bond is a hydroxylated amine, a carboxylate anion or a hydrolase.

12. Composition according to claim 2, in which said compound capable of cleaving at least one ester bond is selected from the group consisting of 3-amino-1,2-propanediol, 2-amino-2-methyl-1,3-propanediol, 2-amino-2-methylpropanol, 2-amino-2-hydroxymethyl-1,3-propanediol, glucamine or N-methylglucamine, a lysine, arginine, and a histidine.

13. Composition according to claim 2, in which said compound capable of cleaving at least one ester bond is a lipase, an esterase or a protease.

14. Composition according to claim 2, in which said compound capable of cleaving at least one ester bond is a lipase.

15. Composition according to claim 2, in which said compound capable of cleaving at least one ester bond is at a concentration ranging from 0.1% to 30% by weight relative to the total weight of the composition.

16. Composition according to claim 2, in which said compound capable of cleaving at least one ester bond is at a concentration ranging from 0.5% to 15% by weight relative to the total weight of the composition.

17. Composition according to claim 2, in which said esterified derivative and said compound capable of cleaving at least one ester bond are packaged so as not to be in contact with each other.

18. Composition according to claim 17, in which said esterified derivative and said compound capable of cleaving at least one ester bond are contained in a single packaging with two compartments.

19. Composition according to claim 18, in which at least one from among the esterified derivatives and said compound capable of cleaving at least one bond is encapsulated.

20. Composition according to claim 1, characterized in that the composition comprises at least one fatty carrier and at least one hydrophilic solvent.

21. Composition according to claim 20, characterized in that the fatty carrier present is one or more compounds chosen from the ceramides, cholesterol, phospholipids, cholesteryl sulphate, cholesteryl phosphate, phosphatidylcholine, lecithin and/or empty liposomes.

22. Composition according to claim 1, characterized in that the formulation comprises one or more UV filters.

23. Composition according to claim 20, characterized in that the formulation comprises, as hydrophilic solvent, glycerol and/or propylene glycol, preferably in a concentration of from 0.1 to 20% by weight.

24. Composition according to claim 20, characterized in that the formulation comprises 0.1 to 10% by weight of fatty carrier.

25. Composition according to claim 1, characterized in that the formulation is liquid, creamy, milky and/or gel-like bath additives, bath tablets, bath salts and/or bath capsules.

26. A method for coloring the skin, consisting in applying thereto a composition according to claim 1.

27. A method for coloring the skin according to claim 26, with application taking place at elevated temperature.

28. A method for coloring the skin according to claim 26, characterized in that application takes place in a bathtub or whirlpool.

29. A method for coloring the skin according to claim 26, characterized in that the application temperature is in the range between 25 and 60° C., preferably between 30 and 55° C. and particularly preferably between 37 and 50° C.

30. A method for coloring the skin according to claim 26, characterized in that application to the skin takes place by misting, preferably by means of a shower or spray gun.

31. Method of tanning the human body, characterized in that at least one esterified dihydroxyacetone derivative corresponding to the general formula I according to claim 1 is dissolved in water, the solution is brought to a temperature which is elevated relative to room temperature and the solution is applied to the human body.

32. Method according to claim 31, characterized in that the human body, as a whole or partially, is immersed into the solution.

33. Method according to claim 31, characterized in that, for even tanning, the human body—completely or partially—is sprayed evenly with the heated solution.

34. Method according to claim 31, characterized in that a water-repellent preparation is applied to parts of the body which are not to be tanned or tanned only slightly.
